(19) United States
(12) Patent Application Publication
(10) Pub. No.: US 2006/0128956 A1
(43) Pub. Date: Jun. 15, 2006

(54) (Purin-6-yl) amino acid and production method thereof

(75) Inventors: Michal Hocek, Praha (CZ); Petr Capek, Praha (CZ)

Correspondence Address: BIRCH STEWART KOLASCH & BIRCH, PO BOX 747, FALLS CHURCH, VA, 22040-0747, US

(73) Assignee: USTAV ORGANICKE CHEMIE A BIOCHEMIE AKADEMIE VED CESKE REPUBLIKY, Praha 6 (CZ)

(21) Appl. No.: 10/537,608

(22) PCT Filed: Mar. 31, 2004
(86) PCT No.: PCT/CZ04/00018

§ 371 Date: Dec. 5, 2005

(30) Foreign Application Priority Data
Apr. 21, 2003 (JP) 2003/115403

(51) Int. Cl. C07D 473/10; C07D 473/12

(52) U.S. Cl. 544/276; 544/277

(57) ABSTRACT

The invention provides a synthetic method of (purin-6-yl)amino acids which are useful as medicaments of anticancer, anti-virus agents and so on or their intermediates. Methyl (R,S)-3-[4-(9-benzylpurin-6-yl) phenyl]-2-[(t-butoxycarbonyl) amino]propanoate is produced by making 9-benzyl-6-iodopurine react with methyl (R,S)-2-[(t-butoxycarbonyl)amino]-3-[4-(trimethyl-stananyl) phenyl]propionate in the presence of Pd₂ dba₃, triphenylarsine and copper iodide.

### TECHNICAL FIELD

The present invention relates to new (purin-6-yl)amino acid and a production method thereof.

### BACKGROUND ART

As an anticancer agent or a compound having an antiviral activity, purine compound is conventionally well known, and there are many reports on synthetic intermediates therefor (e.g., WO00/751588).

An object of the present invention is to provide a new (purin-6-yl)amino-acid which itself is useful as a pharmaceutical product such as an anticancer agent, an antiviral agent and the like, a production intermediate is therefor and a production method thereof.

### SUMMARY OF THE INVENTION

The present invention provides a (purin-6-yl)amino acid represented by the following formula (1) 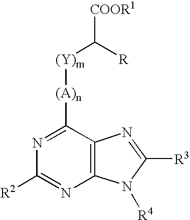
wherein
R¹ is a hydrogen atom, an alkyl group, an optionally substituted aryl group, an optionally substituted heteroaryl group or an aralkyl group;
R² and R³ are each a hydrogen atom, a halogen atom, an optionally substituted alkyl group, an optionally substituted aryl group, an optionally substituted heteroaryl group, an optionally substituted amino group or an optionally substituted hydroxyl group;
R is —NH₂, —NHR′ or —NR′R″;
R′ and R″ are each an amino-protecting group;
Y is alkylene, alkenylene or alkynylene;
A is an optionally substituted phenylene;.
m and n are each 0 or 1; and
R⁴ is a hydrogen atom or an organic group;
   and a salt thereof.

### DETAILED DESCRIPTION OF THE INVENTION

In the (purin-6-yl)amino acid represented by the above formula (1) of the present invention, as the alkyl group for R¹, C1-C15 alkyl group such as methyl group, ethyl group, n-propyl group, iso-propyl group, n-butyl group, iso-butyl group, sec-butyl group, t-butyl group, n-pentyl group, iso-pentyl group, neo-pentyl group, n-hexyl group, heptyl group, octyl group, nonyl group, dodecyl group and the like can be mentioned; as the optionally substituted aryl group, for example, aryl group (e.g., phenyl group, naphthyl group etc.) optionally substituted by the above-mentioned alkyl group, halogen atom (e.g., chlorine atom, bromine atom), nitro group, hydroxyl group, cyano group, carboxyl group and the like can be mentioned, which is specifically exemplified by phenyl group, naphthyl group, tolyl group, xylyl group, 4-oxyphenyl group, 4-chlorophenyl group, 4-nitrophenyl group and the like; and as the optionally substituted heteroaryl group, for example, heteroaryl group (e.g., 2-pyridyl group, 2-quinolyl group, 2-pyrimidyl group, 2-thiophenyl group etc.) optionally substituted by alkyl group, cyano group, carboxyl group, nitro group, halogen atom and the like can be mentioned, and as the aralkyl group, benzyl group and the like can be mentioned.

As the halogen atom for R¹ or R³, fluorine atom, chlorine atom, bromine atom, iodine atom and the like can be mentioned; as the optionally substituted alkyl group, for example, linear, branched or cyclic C1-C15 alkyl group optionally substituted by C7-C15 aralkyloxy group (benzyloxy group, 1-phenethyloxy group, 2-phenethyloxy group etc.), C1-C7 acyl group (acetyloxy group, trimethylacetyloxy group, benzoyloxy group etc.), tri(C1-C7 alkyl)silyloxy group (trimethylsilyloxy group, triethylsilyloxy group, tert-butyldimethylsilyloxy group etc.), (C1-C7 alkyl)oxycarbonyloxy group (tert-butyloxycarbonyl group etc.), C1-C15 alkyloxy group (methoxy group, ethoxy group, n-propoxy group, isopropoxy group etc.) and the like can be mentioned. Specific examples thereof include methyl group, ethyl group, n-propyl group, n-butyl group, iso-butyl group, sec-butyl group, t-butyl group, n-pentyl group, iso-pentyl group, neo-pentyl group, n-hexyl group, n-heptyl group, n-octyl group, n-nonyl group, n-dodecyl group, cyclopropyl group, cyclohexyl group, acetyloxymethyl group, benzyloxymethyl group, methoxymethyl group and the like.

As the optionally substituted aryl group, for example, aryl group (e.g., phenyl group, naphthyl group etc.) which may be substituted by the above-mentioned C1-C15 alkyl group, the above-mentioned halogen atom, nitro group, hydroxyl group, cyano group, carboxyl group and the like, can be mentioned. Specific examples thereof include phenyl group, naphthyl group, tolyl group, xylyl group, 4-oxyphenyl group, 4-chlorophenyl group, 4-nitrophenyl group and the like.

As the optionally substituted heteroaryl group, for example, heteroaryl group such as furyl group, pyrrolyl group, pyridyl group, quinolyl group and the like, which may be substituted by the above-mentioned C1-C15 alkyl group, the above-mentioned halogen atom, nitro group, hydroxyl group, cyano group, carboxyl group and the like, can be mentioned. Specific examples thereof include 4-chloro-2-pyridyl group, 5-bromo-8-quinolyl group and the like.

As the optionally substituted amino group, for example, amino group optionally substituted by C7-C15 aralkyl group (benzyl group, 1-phenethyl group, 2-phenethyl group etc.), C1-C7 acyl group (formyl group, acetyl group, trimethylacetyl group, benzoyl group etc.), tri(C1-C7 alkyl)silyl group (trimethylsilyl group, triethylsilyl group, tert-butyldimethylsilyl group etc.), (C1-C7 alkyl)oxycarbonyl group (tert-butyloxycarbonyl group etc.), C1-C15 alkyl group (methyl group, ethyl group, n-propyl group, isopropyl group etc.) and the like can be mentioned. Specific examples thereof include amino group, benzylamino group, acetylamino group, diacetylamino group, tert-butyloxycarbonylamino group and alkylamino group (methylamino group, ethylamino group, dimethylamino group etc.).

As the optionally substituted hydroxyl group, for example, hydroxyl group optionally substituted by C7-C15 aralkyl group (benzyl group, 1-phenethyl group, 2-phenethyl group etc.), C1-C7 acyl group (acetyl group, trimethylacetyl group, benzoyl group etc.), tri(C1-C7 alkyl)silyl group (trimethylsilyl group, triethylsilyl group, tert-butyldimethylsilyl group etc.), (C1-C7 alkyl)oxycarbonyl group (tert-butyloxycarbonyl group) or C1-C15 alkyl group (methyl group, ethyl group, n-propyl group, isopropyl group etc.) and the like can be mentioned. Specific examples thereof include hydroxyl group, benzyloxy group, acetyloxy group and alkoxy group (methoxy group, ethoxy group etc.).

The substituent R is represented by —NH₂, —NHR′ or —NR′R″, wherein R′ and R″ are amino-protecting groups, and the amino-protecting group is exemplified by C7-C15 aralkyl group (benzyl group, 1-phenethyl group, 2-phenethyl group etc.), C1-C7 acyl group (acetyl group, trimethylacetyl group, benzoyl group etc.), tri(C1-C7 alkyl)silyl group (trimethylsilyl group, triethylsilyl group, tert-butyldimethylsilyl group etc.), (C1-C7 alkyl)oxycarbonyl group (methoxycarbonyl group, ethoxycarbonyl group, t-butoxycarbonyl group etc.), aryloxycarbonyl group (phenoxycarbonyl group etc.) and C1-C15 alkyl group (methyl group, ethyl group, n-propyl group, isopropyl group, nonyl group etc.).

These R′ and R″ may form, for example, benzophenoneimine together with N atom.

In addition, the binding group Y is alkylene, alkenylene or alkynylene, each of which having 1 to 5, preferably 1 to 3, carbon atoms. Here, alkylene, alkenylene and alkynylene are used in a wide sense. For example, alkylene includes methylene, ethylene and the like, and in addition, when the number of carbon is 3 or more, polymethylene such as trimethylene represented by —CH₂CH₂CH₂—, wherein the carbons on both ends of linear hydrocarbon have a free valence.

The binding group A is, for example, phenylene optionally substituted by the above-mentioned C1-C15 alkyl group, the above-mentioned halogen atom, nitro group, hydroxyl group, cyano group, carboxyl group and the like. Specific examples thereof include 2-methyl-1,4-phenylene, 2,6-dimethyl-1,4-phenylene, 5-hydroxy-1,3-phenylene and the like.

In the formula (1), the binding sites of A and Y are specified. In some cases, these binding sites may be inverted, and (Y)ₘ may be bonded on the purine ring side and (A)ₙ may be bonded on the amino acid side.

In the relationship between n and m of (A)ₙ and (Y)ₘ, n and m may be either 0 or 1, wherein the both are mostly 0, or one of them is 0 and the other is 1.

R⁴ shows a hydrogen atom or an organic group. As the organic group, tetrahydropyran-2-yl, 2,3,5-tri-O-acetyl-β-D-ribofuranosyl and the like, as well as the above-mentioned various substituents, amino-protecting group, sugar group and the like can be mentioned.

Here, the sugar group means a structure wherein the carbon atom of the 1-position of the sugar is bonded to nitrogen atom of a purine skeleton. As such sugar, for example, pentose (ribose, arabinose, xylose, lyxose etc.) and hexose (glucose, mannose, galactose, fructose etc.) as well as their deoxy-derivatives (2-deoxyribose, 3-deoxyribose, 5-deoxyribose etc.) can be mentioned. The hydroxyl group of these sugars may be protected by a protecting group. As the protecting group of hydroxyl group of sugar chain, for example, methyl group, acetyl group, benzoyl group, benzyl group, toluyl group, trimethylsilyl group, t-butyldimethylsilyl group and the like can be mentioned.

Preferable embodiments of (purin-6-yl)amino acid of the present invention represented by the formula (1) can be largely divided into three groups.

The first is (purin-6-yl)amino acid represented by the formula (2) 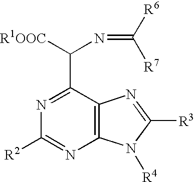
wherein R¹, R², R³ and R⁴ are as defined above, and R⁶ and R⁷ are each a phenyl group. Representative compounds are 9-benzyl-6-{(ethoxycarbonyl)[(diphenylmethylidene)amino]methyl}purine, 9-(tetrahydrofuran-2-yl)-6-{(ethoxycarbonyl)[(diphenylmethylidene)amino]methyl}purine, 9-(2,3,5-tri-β-acetyl-A-D-ribofuranosyl)-6-{(ethoxycarbonyl)[(diphenylmethylidene)amino]methyl}purine and the like.

The second is (purin-6-yl)amino acid represented by the formula (3) 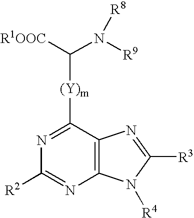
wherein R¹, R², R¹, R⁴, Y and m are as defined above, and R⁸ and R⁹ each may be a hydrogen atom or an amino-protecting group.

Here, the amino-protecting group of R⁸ and R⁹ is the same as the aforementioned amino-protecting group.

Of the (purin-6-yl)amino acids represented by this formula (3), (purin-6-yl)amino acid wherein (Y)ₘ is alkylene, particularly methylene, (purin-6-yl)amino acid, wherein (Y)ₘ is trimethylene, which is represented by the following formula (4) 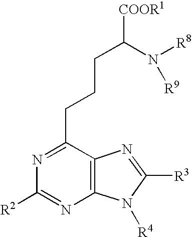
wherein R¹, R², R³, R⁴, R⁸ and R⁹ are as defined above, and (purin-6-yl)amino acid wherein (Y)ₘ is ethynylene represented by —C≡C— are representative amino acids.

Here, specific examples of the aforementioned (purin-6-yl)amino acid wherein (Y)ₘ is methylene include benzyl (R,S)-3-(9-benzylpurin-6-yl)-2-[(t-butoxycarbonyl)amino]propanoate, and examples of the (purin-6-yl)amino acid wherein (Y)ₘ is trimethylene include ethyl (R,S)-2-amino-5-(9-benzylpurin-6-yl)pentanoate.

The third is (purin-6-yl)amino acid represented by the formula (5) 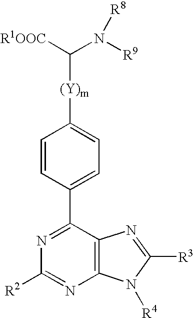
wherein R¹, R², R³, R⁴, R⁸, R⁹, Y and m are as defined above.

Here, a compound wherein (Y)ₘ is alkylene, particularly methylene, is representative, and as such compound, methyl (R,S)-3-[4-(9-benzylpurin-6-yl)phenyl]-2-[(t-butoxycarbonyl)amino]propanoate can be mentioned.

As the salt of the compound represented by the formula (1) of the present invention, for example, salts with inorganic acids, salts with organic acids, salts with inorganic bases, salts with organic bases, salts with amino acids and the like can be mentioned. Examples of preferable salts with inorganic acids include hydrochloride, hydrobromide, sulfate and the like. Examples of preferable salts with organic acids include acetate, trifluoroacetate, tartrate, methanesulfonate and the like. Examples of preferable salts with inorganic bases include alkali metal salts (e.g., sodium salt etc.), alkaline earth metal salts (e.g., calcium salt etc.) and the like. Examples of preferable salts with organic bases include trimethyl amine salt, triethyl amine salt, pyridine salt and the like. Examples of preferable salts with amino acids include lysine salt, aspartate and the like.

The production methods of (purin-6-yl)amino acid of the present invention are described in the following for every group described above.

The above-mentioned (purin-6-yl)amino acid represented by the formula (2) can be produced by reacting a halogenated purine compound represented by the formula (6) 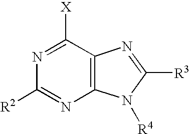
wherein X is a halogen atom, and R², R³ and R⁴ are as defined above, with an amino acid derivative represented by the formula (7) 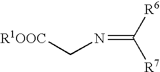
wherein R¹, R⁶ and R⁷ are as defined above.

In both starting compounds represented by the formulas (6) and (7), each substituent of R², R³, R⁴, R¹, R⁶ and R⁷ is required to have a substituent corresponding to the substituent that the object compound represented by the formula (2) has. For both compounds, the substituent X is particularly preferably iodine atom, from among halogen atoms such as chlorine atom, bromine atom, iodine atom and the like, from the aspect of reactivity.

Of such starting compounds, as the halogenated purine compound represented by the formula (6), for example, 9-benzyl-6-iodoprine, 9-(tetrahydrofuran-2-yl)-6-iodoprine, 9-(2,3,5-tri-O-acetyl-β-D-ribofuranosyl)-6-iodoprine, and chlorides and bromides instead of iodides of these can be mentioned. From the aspect of reactivity, iodide is preferable.

As the amino acid derivative represented by the formula (7), moreover, glycine derivative, such as ethyl [(diphenylmethylidene)amino]acetate, t-butyl [(diphenylmethylidene)amino]acetate and the like can be mentioned.

This method is performed in the presence of a base and a palladium catalyst and conventionally in a solvent. As the base, carbonate, phosphate, hydride and the like of alkali metal and alkaline earth metal can be mentioned. Specific examples thereof include potassium carbonate, sodium carbonate, magnesium carbonate, potassium phosphate, sodium phosphate, sodium hydride and the like, with preference given to potassium phosphate.

As the palladium catalyst, organic acid salts of palladium, acetates such as Pd(OAc)₂ and the like, coordination compounds of palladium and dibenzylideneacetone and the like such as Pd(dba)₂ and Pd₂(dba)₃ and the like, can be mentioned.

In addition, it is preferable to use the palladium catalyst in the form of a palladium complex using, as a ligand, organic phosphines such as tri-t-butylphosphine (t-Bu₃P), dicyclohexylbiphenylphosphine (Cy₂biphen), triphenylphosphine (PPh₃) and the like or an iron complex (dppf) consisting of two molecules of cyclopentadienyldiphenylphosphine and one molecule of iron.

In this reaction, the amount to be used of the amino acid derivative represented by the formula (7) is not less than an equimolar amount, generally 1 to 3-fold equivalents, relative to the halogenated purine compound represented by the formula (6), which is the other starting material.

The amount of the base to be used is generally 0.1 to fold equivalents, preferably 1 to 5-fold equivalents, relative to the halogenated purine compound represented by the formula (6), and the amount of the palladium catalyst to be used is generally 0.005 to 1-fold equivalent, preferably. 0.01 to 0.2-fold equivalent, relative to the halogenated purine compound. In addition, a phosphine ligand is used in an amount of generally 0.005 to 1-fold equivalent, preferably 0.01 to 0.5-fold equivalent, relative to the halogenated purine compound.

The solvent is not particularly limited as long as it is inert to the reaction and exemplified by dimethylformamide, tetrahydrofuran, dioxane, toluene, benzene and the like. The amount of the solvent to be used is generally 1 to 100-fold weight, preferably 5 to 50-fold weight, relative to the halogenated purine compound represented by the formula (6).

The reaction temperature at this time is optional as long as it is not higher than the boiling point of the organic compound in the reaction system. It is generally 10-150° C., preferably 50-120° C., and the reaction time is generally 1-48 hours, preferably 2-24 hours.

The above-mentioned (purin-6-yl)amino acid represented by the formula (3) can be produced by reacting the above-mentioned halogenated purine compound represented by the formula (6) with a halogenated amino acid derivative represented by the formula (8) 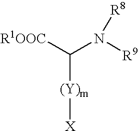
wherein R¹, R⁸, R⁹, X, Y and m are as defined above.

In both starting compounds represented by, the formulas (6) and (8), each substituent of R², R³, R⁴, R¹, R⁸ and R⁹ is required to have a substituent corresponding to the substituent that the object (purin-6-yl)amino acid represented by the formula (3) has. For both compounds, the substituent X is particularly preferably iodine atom, from among halogen atoms such as chlorine atom, bromine atom, iodine atom and the like, from the aspect of reactivity.

In the above-mentioned reaction, a compound represented by the formula (6), which is a starting material, is as defined above, and as a halogenated amino acid derivative represented by the formula (8), which is the other starting material, benzyl (R,S)-2-[(t-butoxycarbonyl)amino]-3-iodopropanoate, benzyl (R,S)-2-[(t-ethoxycarbonyl)amino]-3-bromopropanoate and the like can be mentioned.

Generally in this reaction, a halogenated amino acid derivative represented by the formula (8), which is a starting material, is reacted with zinc (Zn) to give a zinc compound, wherein —X has been converted to —ZnX in the formula (8) (Step 1), and the reaction product is reacted with a halogenated purine compound represented by the formula (6) (Step 2).

The reaction of this Step 1 is carried out in the presence of trialkylsilyl halide (e.g., as trimethylsilyl chloride etc.) in an organic solvent.

The amount of zinc powder to be used is generally 3 to 15-fold equivalents, preferably 4 to 10-fold equivalents, relative to the halogenated amino acid derivative, which is a starting material, and the amount of trialkylsilyl halide to be used is generally 0.01 to 1-fold equivalent, preferably 0.05 to 0.5-fold equivalent, relative to the halogenated amino acid derivative.

For a solvent, dimethylformamide, tetrahydrofuran, dioxane and the like are used. The amount of the solvent to be used is generally 1 to 100-fold weight, preferably 5 to 50-fold weight, relative to the halogenated amino acid derivative.

The reaction method generally includes dispersing a zinc powder and trialkylsilyl halide in a solvent such as dimethylformamide and the like, sonicating the mixture, adding thereto a solution of a halogenated amino acid derivative represented by the formula (8) in a solvent such as tetrahydrofuran, sonicating the mixture, and removing the remaining zinc powder.

The reaction temperature at this time is optional as long as it is not higher than the boiling point of the organic compound in the reaction system. It is generally 10-150° C., and the reaction time is generally 1-48 hours, preferably 2-24 hours.

The reaction of Step 2 is carried out by reacting the zinc compound produced in Step 1 with a halogenated purine compound represented by the formula (6) in a solvent in the presence of a palladium catalyst.

In this Step, as a solvent, the solvents similar to those used in Step 1 can be mentioned, and as a palladium catalyst, the palladium catalysts similar to those mentioned above are used.

The amount to be used of the halogenated amino acid derivative represented by the formula (8) is generally not less than an equimolar amount, preferably 1 to 3-fold equivalents, relative to the halogenated purine compound represented by the formula (6), which is the other starting material. The amount of the palladium catalyst to be used is generally 0.005 to 1-fold equivalent, preferably 0.01 to 0.2-fold equivalent, relative to the halogenated purine compound. In addition, a phosphine ligand is used in an amount of generally 0.005 to 1-fold equivalent, preferably 0.01 to 0.5-fold equivalent, relative to the halogenated purine compound. The amount of the solvent to be used is generally 1 to 100-fold weight, preferably 5 to 50-fold weight, relative to the halogenated purine compound.

The reaction method includes adding the above-mentioned halogenated purine compound represented by the formula (6), which is a starting material, a palladium catalyst, and preferably a ligand similar to the above-mentioned one to a solvent such as dimethylformamide, adding this to the reaction solution after removing the zinc powder obtained in Step 1, and stirring the mixture to allow for reaction.

The reaction temperature at this time is optional as long as it is not higher than the boiling point of the organic compound in the reaction system. It is generally 10-150° C., and the reaction time is generally 1-48 hours, preferably 2-24 hours.

Of the above-mentioned (purin-6-yl)amino acids represented by the formula (3), a compound represented by the formula (4), wherein (Y)ₘ is trimethylene, can be also produced by reacting the aforementioned halogenated purine compound represented by the formula (6) with an amino acid derivative represented by the following formula (9) 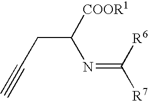
wherein R¹, R⁶ and R⁷ are as defined above.

As the amino acid derivative represented by the formula (9), which is a starting material, a compound having a substituent corresponding to the object (purin-6-yl)amino acid represented by the formula (4) is used. For example, ethyl (R,S)-2-[(diphenylmethylidyne)amino]pent-4-enoate can be mentioned.

In this reaction, the halogenated purine compound represented by the formula (6) is subjected to a coupling reaction with an amino acid derivative represented by the formula (9) by a well-known method to give a compound of the above-mentioned formula (3) wherein (Y)ₘ is ethynylene, which compound is then hydrogenated to easily give (purin-6-yl)amino acid wherein (Y)ₘ is trimethylene.

The coupling reaction is usually carried out in a solvent in the presence of a metal halide, a palladium catalyst and an organic base.

As the metal in the metal halide, copper is representative, and as halogen, iodine atom is representative.

As the palladium catalyst, a catalyst similar to the above-mentioned one is used, and as the organic base, organic amines, such as triethylamine, can be mentioned.

In this reaction, the amount to be used of the amino acid derivative represented by the formula (9) is not less than an equimolar amount, generally 1 to 3-fold equivalents, relative to the halogenated purine compound represented by the formula (6), which is the other starting material.

The amount of the metal halide to be used is generally 0.05 to 1-fold equivalent, preferably 0.08 to 0.5-fold equivalent, relative to the halogenated purine compound represented by the formula (6). The amount of the palladium catalyst to be-used is generally 0.005 to 0.1-fold equivalent relative to the halogenated purine compound represented by the formula (6).

As the solvent, dimethylformamide, tetrahydrofuran and the like are used. The amount of the solvent to be used is generally 1 to 100-fold weight, preferably 5 to 50-fold weight, relative to the halogenation purine compound.

The reaction temperature at this time is optional as long as it is not higher than the boiling point of the organic compound in the reaction system. It is generally 10-150° C., and the reaction time is generally 1-48 hours, preferably 2-24 hours.

The hydrogenation reaction is carried out by reacting the coupling reaction product obtained by the above-mentioned reaction with hydrogen according to conventional hydrogenation reaction in the presence of a catalyst generally under pressurization.

As the catalyst here, for example, Pd/C and palladium chloride well-known as hydrogenation catalysts can be mentioned. The amount thereof to be used is 0.05 to 0.5-fold weight relative to the above-mentioned coupling reaction product.

While the solvent for the coupling reaction is not particularly limited as long as it is inert to the reaction, alcohols such as methanol, ethanol and the like is preferably used. The amount of the solvent to be used is generally 1 to 100-fold weight, preferably 5 to 50-fold weight, relative to the above-mentioned coupling reaction product.

The reaction time is generally 1-48 hours, preferably 2-24 hours.

The above-mentioned (purin-6-yl)amino acid represented by the formula (5) can be produced by reacting the above-mentioned halogenated purine compound represented by the formula (6) with an amino acid derivative represented by the following formula (10) 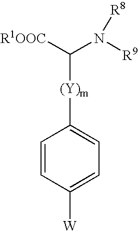
wherein R¹, R⁸, R⁹; Y and m are as defined above, W is —Sn(R⁵)₃, and R⁵ is a lower alkyl group.

In the amino acid derivative represented by the formula (10), which is a starting material for this reaction, the substituent R⁵ is exemplified by methyl, ethyl, propyl and the like, and as the amino acid derivative, methyl (R,S)-2-[(t-butoxycarbonyl)amino]-3-[4-(trimethylstannanyl)phenyl]propionate and the like can be mentioned.

The reaction of a halogenated purine compound represented by the formula (6) with an amino acid derivative represented by the formula (10) is generally carried out in a solvent in the presence of a metal halide, a palladium catalyst and an arsenic compound.

Here, the metal halide and palladium catalyst are as defined above, and as the arsenic compound, trialkylarsine such as trimethylarsine, triphenylarsine and the like and triarylarsine can be mentioned.

In this reaction, the amount to be used of an amino acid derivative represented by the formula (10) is generally 1 to 3-fold equivalents, preferably 1.05 to 1.5-fold equivalents, relative to the halogenated purine compound represented by the formula (6).

The amount of the metal halide to be used is generally 0.05 to 1-fold equivalent, preferably 0.08 to 0.5-fold equivalent, relative to the halogenated purine compound represented by the formula (6). The amount of the palladium catalyst to be used is generally 0.005 to 0.1-fold equivalent, and the amount of the arsenic compound to be used is 0.05 to 0.5-fold equivalent, preferably 0.08 to 0.3-fold equivalent.

As the solvent, dimethylformamide, tetrahydrofuran and., the like are used. The amount of the solvent to be used is generally 1 to 100-fold weight, preferably 2 to 50-fold weight, relative to the halogenated purine compound.

The reaction temperature at this time is optional as long as it is not higher than the boiling point of the organic compound in the reaction system. It is generally 10-100° C., and the reaction time is generally 1-100 hours, preferably 2-50 hours.

After the completion of the reaction, a catalyst is removed according to conventional methods and then the solvent is removed according to conventional methods to isolate the object compound, after which the compound is purified as necessary by an appropriate method to give the object compound.

When the object compound is obtained as a free base, it can be converted to the object salt by a method known per se or a method analogous thereto. When it is obtained as a salt, it can be converted to a free base or other object salt by a method known per se or a method analogous thereto.

When the object compound has stereoisomers, they can be also isolated as desired by appropriate separation and purification methods. When the object compound is a racemate, it can be separated into an S form and an R form by conventional methods for optical resolution. Moreover, it is also possible to produce the object compound in an S form or R form using an (S)- or (R)-halogenated amino acid derivative as a starting material. When the object compound has a stereoisomer, the present invention encompasses a single isomer and a mixture thereof.

The (purin-6-yl)amino acid represented by the formula (1) can be easily produced by such methods and the obtained (purin-6-yl)amino acid is useful as a pharmaceutical product such as an anti-cancer agent, an antiviral agent and the like or a production intermediate therefor.

### EXAMPLES

The present invention is be explained in more detail in the following by referring to Examples. It is needless to say that these Examples are not intended to restrict the present invention.

### Example 1-1

### Production of benzyl(R,S)-3-(9-benzylpurin-6-yl)-2-[(tert-butoxycarbonyl)amino]propanoate

Dimethylformamide (DMF) (1 ml) containing zinc powder (380 mg, 6 mmol) dispersed therein was placed in a flask and the flask was purged with an argon gas. Thereto was added trimethylsilyl chloride. (80 μl, 0.6 mmol). This mixture was sonicated at room temperature for 30 min and then a solution of benzyl (R,S)-2-[(tert-butoxycarbonyl)amino]-3-iodopropanoate (405 mg, 1 mmol) in DMF (3 ml) was continuously added under an argon atmosphere, which was followed by sonication at room temperature for 40 min. This was transferred into a solution of 9-benzyl-6-iodopurine (168 mg, 0.5 mmol), Pd₂ dba₃ (17 mg, 0.02 mmol) and tri(o-tolyl)phosphine (23 mg, 0.08 mmol) in DMF (2 ml).

The reaction mixture was stirred at room temperature for 5 hr and left standing overnight.

Thereafter, the solvent was evaporated under reduced pressure, and the residue was diluted with ethyl acetate (70 ml) and washed twice with water (60 ml each) and once with brine (60 ml).

The solvent was evaporated from the organic layer and the residue was purified by silica gel column (ethyl acetate/hexane, 1:1) chromatography to give benzyl (R,S)-3-(9-benzyl(purin-6-yl))-2-[(tert-butoxycarbonyl)amino]propanoate (216 mg, yield: 89%) as a colorless, amorphous solid.

### Example 1-2

### Production of benzyl (R,S)-3-(9-benzylpurin-6-yl)-2-[(tert-butoxycarbonyl)amino]propanoate

Trimethylsilyl chloride (160 μl, 1.3 mmol) was added through septum- to an argon purged flask containing a suspension of zinc powder (2.8 g, 43 mmol) in DMF (4 ml). The mixture was sonicated at room temperature for 20 min. Then a solution of benzyl (R,S)-2-[(tert-butoxycarbonyl)amino]-3-iodopropanoate (2.9 g, 7.2 mmol) in DMF (22 ml) prepared under argon was added through septum to the suspension of activated Zn and the sonication was continued for another 40 min at room temperature, after which zinc was allowed to settle. The supernatant was transferred through septum to a mixture of 9-benzyl-6-iodopurine (1.35 g, 4.0 mmol), Pd₂ dba₃ (104 mg, 0.12 mmol) and tri(o-tolyl)phosphine (146 mg, 0.48 mmol) in DMF (16 ml) prepared under argon. The reaction mixture was stirred at room temperature for 8 hr and allowed to stay overnight and then the solvent was evaporated in vacuo. The residue was diluted with ethyl acetate (70 ml) and washed with water (2×60 ml) and brine (60 ml). The organic phase was evaporated and the residue was chromatographed on a silica gel column (ethyl acetate/hexane, 1:1) to give the title compound (1.85 g, yield 95%) as a colorless, amorphous solid.

MS (FAB): 488 (6, M+1); 432 (5); 252 (8); 225-(9); 147 (14); 91 (Bn).

HRMS (FAB): for C₂₇H₃₀N₅O₄ calculated 488.2298; found 488.2290.

¹H NMR (500 MHz, CDCl₃): 1.40 (s, 9H, 3×CH₃-Boc); 3.62 (dd, 1H, J=4.6 and 15.8, CH_{A}H_{B}CH); 3.89 (dd, 1H, J=5.7 and 15.8, CH_{A}H_{B}CH); 4.98 (m, 1H, CHCO); 5.09 (dd, 2H, J=11.9 and 38.7, OCH₂Ph); 5.40 (s, 2H, NCH₂Ph); 6.16 (d, 1H, J=8.5, NH); 7.20-7.38 (m, 10H, arom.); 7.97 (s, 1H, H-8); 8.80 (s, 1H, H-2).

¹³C NMR (125.8 MHz, CDCl₃): 28.25 (CH₃); 34.58 (Pu—CH₂); 47.26 (NCH₂Ph); 51.73 (CHCO); 66.93 (OCH₂Ph); 79.71 (C(CH₃)₃); 127.86, 128.00, 128.04, 128.26, 128.62 and 129.13 (CH-arom.); 132.69. (C-5); 134.96 and 135.44 (C-arom.); 143.90 (CH-8); 150.75 (C-4); 152.19 (CH-2); 155.53 (CO-Boc); 157.72 (C-6); 171.57 (COOBn).

IR (CHCl₃): 3436, 3096, 3033, 3011, 2983, 1744, 1710, 1598, 1499, 1456, 1406, 1368, 1334, 1230, 1193, 1163, 1057, 1028.

### Example 2-1

### Production of methyl(R,S)-3-[4-(9-benzylpurin-6-yl)phenyl]-2-[(tert-butoxycarbonyl)amino]propanoate

DMF (15 ml) was added to a flask containing 9-benzyl-6-iodopurine (547 mg, 1.65 mmol), methyl(R,S)-2-[(tert-butoxycarbonyl)amino]-3-[4-(trimethylstannanyl)phenyl]propionate (827 mg, 1.87 mmol), Pd₂ dba₃ (92 mg, 0.1 mmol), triphenylarsine (AsPh₃) (61 mg, 0.2 mmol) and copper iodide (CuI)(76 mg, 0.4 mmol) under an argon atmosphere, and the mixture was stirred at 80° C. for 30 hr.

The solvent was evaporated under reduced pressure from the reaction mixture and the residue was dissolved in ethyl acetate. The ethyl acetate solution was washed with water (80 ml) and brine (80 ml).

The solvent was evaporated from the organic layer and the residue was purified by silica gel column (ethyl acetate/hexane, 1:2) chromatography to give a crude product. This was recrystallized from ethyl acetate/hexane to give the title compound (360 mg, yield: 45%) as white crystals.

### Example 2-2

### Production of methyl (R,S)-3-[4-(9-benzylpurin-6-yl)phenyl]-2-[(tert-butoxycarbonyl)amino]propanoate

DMF (15 ml) was added through septum to an argon purged flask containing 9-benzyl-6-iodopurine (547 mg, 1.65 mmol), methyl (R,S)-2-[(tert-butoxycarbonyl)amino]-3-[4-(trimethylstannanyl)phenyl]propionate (827 mg, 1.87 mmol); Pd₂ dba₃ (92 mg, 0.1 mmol), AsPh₃ (61 mg, 0.2 mmol) and CuI (125 mg, 0.66 mmol). The mixture was stirred at 80° C. for 24 hr. The solvent was evaporated in vacuo, the residue was dissolved in ethyl acetate (80 ml) and washed with water (80 ml), filtrated and washed with brine (80 ml). The organic phase was evaporated and the residue was chromatographed on a silica gel 1 column (ethyl acetate/hexane, 1:2) and recrystallized from ethyl acetate/heptane to give the title compound (440 mg, yield 55%) as white crystals.

m.p. 133-136° C.

MS (FAB): 488 (6, M+1); 432 (18); 337 (6); 300 (8); 91 (100, Bn).

HRMS (FAB): for C₂₇H₃₀N₅O₄ calculated 488.2298, found 488.2309.

¹H NMR (500 MHz, CDCl₃): 1.43 (s, 9H, 3×CH₃-Boc); 3.19 (m, 2H, CH₂CH); 3.72 (s, 3H, OCH₃); 4.65 (dd, 1H, J=13.4 and 5.6, CHNH); 5.05 (d, 1H, J=8.7, NH); 5.48 (s, 2H, CH₂Ph); 7.31-7.37 (m, 7H, arom.); 8.10 (s, 1H, H-8); 8.74 (d, 2H, J(CH-arom., CH-arom.)=8.3, 2×CH-arom.); 9.04 (s, la, H-2). ¹³C NMR (125.8 MHz, CDCl₃): 28.26 ((CH₃)₃); 38.13 (CH₂CH); 47.23 (CH₂Ph); 52.23 (OCH₃); 54.27 (CHNH); 79.95 (C(CH₃)₃); 127.76, 128.54, 129.11, 129.63 and 129.91 (5×CH-arom.); 130.80 (C-5); 134.47 (C-1-arom.); 135.15 (C-1-Ph); 139.20 (C-p-arom.); 144.06 (CH-8); 152.49 (C-4); 152.55 (CH-2); 154.47 (C-6); 155.05 (COO from Boc); 172.07 (COOMe).

IR (CHCl₃): 3438, 1743, 1710, 1583, 1561, 1498, 1450, 1249, 1165, 1063.

Anal. calculated for C₂₇H₂₉N₅O₄ (487.6): C, 66.51%; H, 6.00%; N, 14.36%; found: C, 66.52%; H, 5,94%; N, 14.23%

### Example 3

### Production of ethyl (R,S)-2-[(diphenylmethylidene)amino]-5-(9benzylpurin-6-yl)pent-4-ynoate

DMF (20 ml) and Et₃N were added through septum to an argon purged flask containing 9-benzyl-6-iodopurine (3.03 g, 9 mmol), ethyl (R,S)-2-[(diphenylmethylidene) amino]pent-4-ynoate (3.57 g, 11.7 mmol), CuI (200 mg, 1.05 mmol) and Pd(PPh₃)₄ (300 mg, 0.26 mmol). The mixture was stirred at 65° C. for 7 hr. The solvent was evaporated in vacuo and isolated by column chromatography on silica gel (ethyl acetate/hexane, 4:3) to give the title compound (3.85 g, 83%) as a yellowish amorphous solid.

MS (EI): 513 (6, M); 440 (33); 436 (35, M-Ph); 426 (14); 333 (12, M-N═CPh₂); 266 (7); 193 (15); 180 (9); 165 (18); 104 (6); 91 (100, Bn).

HRMS (EI): for C₃₂H₂₇N₅O₂ calculated 513.2165, found 513.2192.

¹H NMR (500 MHz, CDCl₃): 1.27 (t, 3H, J(CH₃, CH₂)=7.1, CH₃); 3.18 (dd, 1H, J=17.1 and 8.5, CHAHBC≡C); 3.32 (dd, 1H, J=17.1 and 4.9, CH_{A}H_{B}C≡C); 4.14-4.27 (m, 2H, CH₂CH₃); 4.49. (dd, 1H, J=8.5 and 4.9, CHCO); 5.41 (s, 2H, CH₂Ph); 7.26-7.43 (m, 13H, arom.); 7.67 (d, 2H, J=7.4, arom.); 8.01 (s, 1H, H-8); 8.90 (S, 1H, H-2).

¹³C NMR (125.8 MHz, CDCl₃): 14.01, (CH₃); 24.65 (CH₂C≡C); 47.29 (CH₂Ph); 61.39 (OCH₂); 63.86 (CHCO); 77.60 (C≡C—Pu); 97.42 (CH₂—C≡C); 127.79, 127.92, 128.31, 128.45, 128.63, 128.65, 129.03, 129.14 and 130.37 (CH-arom.); 134.27 (C-5); 135.97 and 139.40 (C-arom.); 142.00 (C-6); 144.68 (CH-8); 151.47 (C-4); 152.63. (CH-2); 170.27 (COO); 172.35 (CH-Ph₂) IR (CHCl₃): 2238, 1734, 1624, 1583, 1498, 1447, 1329, 1240, 1195.

### Example 4

### Production of ethyl (R,S)-2-amino-5-(9-benzylpurin-6-yl)pentanoate

Ethyl (R,S)-2-[(diphenylmethylidene)amino]-5-(9-benzylpurin-6-yl)pent-4-ynoate (650 mg, 1.27 mmol) obtained in Example 3 in ethanol (80 ml) was hydrogenated under slight overpressure in the presence of Pd/C catalyst (10 wt %, 80 mg). PdCl₂ (10% solution in 1 M aq. HCl, 100 μl) was added through septum after 15 min and the hydrogenation was continued for 10 hr (the progress was monitored by TLC). The catalyst was filtered off through Celite pad and the filtrate was evaporated in vacuo. The residue was chromatographed on a silica gel column (ethyl acetate/methanol, 19:1 to 9:1) to give the title compound (250 mg, 56%) as a brown amorphous solid.

MS (FAB): 354 (21, M+1); 237 (14); 149 (17); 91 (100, Bn).

¹H NMR (200 MHz, CDCl₃): 8.91 (s, 1H, H-2); 8.04 (s, 1H, H-8); 7.36-7.29 (m, 5H, arom.); 5.44 (s, 2H, CH₂Ph); 4.16 (q, 2H, J(CH₂, CH₃)=7.1, OCH₂); 3.58 (br t, 1H, J=6.2, CHNH); 3.25 (t, 2H, J=7.2, Pu—CH₂CH₂); 3.00 (br, 2H, NH₂); 2.09-1.68 (m, 4H, 2×CH₂); 1.24 (t, 3H, J(CH₃, CH₂)=7.1, CH₃).

### Example 5

### Production of ethyl (R,S)-2-amino-5-(9-benzylpurin-6-yl)pent-4-ynoate

20% Aqueous citric acid (9 ml) was added to a solution of ethyl (R,S)-2-[(diphenylmethylidene)amino]-5-(9-benzylpurin-6-yl)pent-4-ynoate (752 mg, 1.47 mmol) obtained in Example 3 in THF (18 ml) and the mixture was stirred at ambient temperature for 1 h. Then the reaction mixture was diluted with water (70 ml) and washed with ethyl acetate (2×70 ml). To the water phase was added a saturated solution of NaHCO₃ (20 ml) to make pH basic, then the solution was washed with ethyl acetate (2×60 ml) and the obtained organic phase was evaporated. The residue was chromatographed on a silica gel column (ethyl acetate/methanol, 17:3) to give the product (328 mg, 64%) as a yellow amorphous solid.

MS (EI): 349 (3, M); 347 (3); 276 (41, M−COOEt); 248 (100, M−glycinyl+H); 223 (9); 158 (8); 91 (95, Bn).

HRMS (EI): for C₁₉H₁₉N₅O₂ calculated 349.1539, found 349:1544

¹H NMR (200 MHz, CDCl₃): 8.94 (s, 1H, H-2); 8.07 (s, 1H, H-8); 7.39-7.27 (m, 5H, arom.); 5.45 (s, 2H, CH₂Ph); 4.24 (q, 2H, J(OCH₂, CH₃)=7.1, OCH₂); 3.82 (m, 1H, CH—NH₂); 3.10 (dd, IR, J=17.1 and 4.9, CH_{A}H_{B}C≡C); 2.95 (dd, 1H, J 17.1 and 7.4, CH_{A}H_{B}C≡C); 1.94 (br s, 2H, NH₂); 1.29 (t, 3H, J(CH₃, OCH₂) 7.1, CH₃).

¹³C NMR (50.3 MHz, CDCl₃): 173.35 (CO); 152.68 (CH-2); 151.53 (C-4); 144.98 (CH-8); 141.72 (C-6); 134.81 (C-5); 134.46 (C-arom.); 129.17 (2C, CH-arom.); 128.68 (CH-arom.); 127.80 (2C, CH-arom.); 96.09 (CH₂—C≡C); 78.36 (C≡C—Pu); 61.41 (OCH₂); 53.26 (CH—NH₂); 47.35 (CHH₂Ph); 26.55 (CH₂—C≡); 14.14 (CH₃). IR (CHCl₃): 3387, 2239, 1735, 1621, 1584, 1404, 1242, 1197.

### Example 6

### Production of (R,S)-9-benzyl-6-[(diphenylmethylideneamino) (ethoxycarbonyl)methyl]purine

Toluene was added to a flask containing 9-benzyl-6-iodopurine (1 mmol), ethyl [(diphenylmethylidene)amino]acetate (374 mg, 1.28 mmol), potassium phosphate (2 mmol) as a base, Pd(OAc)₂ (22 mg, 0.1 mmol) and tri-t-butylphosphine (0.2 mmol) as a phosphine ligand under an argon atmosphere, and the mixture was stirred at 100° C. for 10 hr.

After the completion of the reaction, the solvent was evaporated and the residue was purified by silica gel column (ethyl acetate/hexane, 1:2-5:1) chromatography to give (R,S)-9-benzyl-6-[(diphenylmethylideneamino) (ethoxycarbonyl)methyl]purine as colorless crystals (yield: 32%).

m.p.: 189-192° C.

MS (FAB): 476

HRMS (EI): for C₂₉H₂₆N₅O₂ calculated 476.2087, found 476.2068. ¹H NMR (500 MHz, CDCl₃): 1.17 (t, 3H, J=7.1, CH₃CH₂); 4.20 (d, 2H, J=7.1, CH₃CH₂); 5.40 (d, 1H, J_{gem}=≈15.1, CH₂Ph-a), 5.45 (d, 1H, J_{gem}=15.1, CH₂Ph-b); 6.01 (s, 1H, COCHN); 7.26-7.44 (m, 13H, H-arom.); 7.71 (d, 2H, J=7.5, H-arom.); 8.00 (s, 1H, H-8); 9.01 (s, 1H, H-2)

¹³C NMR (100.6 MHz, CDCl₃): 14.05 (CH₃); 47.29 (CH₂Ph); 61.54 (CH₂CH₃); 67.62 (COCHN); 127.90, 127.95, 128.55, 128.58, 128.81, 129.11, 129.25, 130.51 (CH-arom.); 132.22, 135.08, 136.10, 139.42 (C-5, C-1-arom.); 144.34 (CH-8); 151.94 (C-4); 152.69 (CH-2); 157.33 (C-6); 169.27 (C═O); 172.75 (C═N)

### Example 7 to 11

### Production of (R,S)-9-benzyl-6-[(diphenylmethylideneamino)(ethoxycarbonyl)methyl]purine

A solvent shown in Table 1 was added to a flask containing 9-benzyl-6-iodopurine (1 mmol), ethyl [(diphenylmethylidene)amino]acetate (374 mg, 1.28 nmol), a base (2 mmol) shown in Table 1, Pd(OAc)₂ (22 mg, 0.1 mmol) and tri-t-butylphosphine (0.2 mmol) as a phosphine ligand shown in Table 1 (0.2 mmol for 1-coordinate ligand and 0.1 mmol for 2-coordinate ligand) under an argon atmosphere, and the mixture was stirred at 100° C. for the reaction time as shown in Table 1. After the completion of the reaction, the reaction mixture was treated in the same manner as in Example 6 to give (R,S)-9-benzyl-6-[(diphenylmethylideneamino)(ethoxycarbonyl)methyl]purine as colorless crystals in a yield shown in Table 1.

TABLE 1

| | | | | reaction | yield |
|---|---|---|---|---|---|
| No. | ligand | base | solvent | time (hr) | (%) |
| 7 | Cy₂Pbiphen | K₃PO₄ | toluene | 28 | 16 |
| 8 | Cy₂Pbiphen | K₃PO₄ | dioxane | 14 | 37 |
| 9 | dppf | K₃PO₄ | dioxane | 14 | 32 |
| 10 | Cy₂Pbiphen | NaH | toluene | 28 | 7 |
| 11 | Cy₂Pbiphen | K₃PO₄ | DMF | 8 | 55 |

### Example 12

### Production of 6-[(R,S)-(diphenylmethylideneamino) (ethoxycarbonyl)methyl]-9-[(R,S)-(tetrahydropyran-2-yl)]purine

The reaction under the same conditions as in Example 11 except that 9-(tetrahydropyran-2-yl)-6-iodopurine (1 mmol) was used instead of 9-benzyl-6-iodopurine (1 mmol), followed by working up gave 6-[(R,S) (diphenylmethylideneamino) (ethoxycarbonyl)methyl]-9-[(R,S)-(tetrahydropyran-2-yl)]purine as a yellow amorphous solid (yield 63s).

MS (FAB): 470

HRMS (EI): for C₂₉H₂₈N₅O₃ calculated 470.2192, found 470.2178.

¹H NMR (400 MHz, CDCl₃): 1.16 (t, 3H, J=7.1, CH₃CH₂); 1.64-2.15 (m, 6H, CH₂-THP); 3.79, (brt, 1H, J=10.8, CH₂-Oa); 4.16-4.22 (m, 3H, CH₂CH₃, CH₂-Ob); 5.80 (d, 1H, J=9.8, OCHN); 5.98, 6.00 (2×s, 2×½H, NCHCO); 7.26-7.72 (m, 10H, H-arom); 8.25 (9, 1H, H-8); 8.98 (s, 1H, H-2)

¹³C NMR (100.6 MHz, CDCl₃): 14.04 (CH₃CH₂); 22.75, 24.83, 31.74 (CH₂-THP); 61.59. (CH₂CH₃); 67.66 (COCHN); 68.81 (CH₂—O); 81.95 (NCHO); 127.90, 128.24, 128.58, 128.85, 129.26, 130.53 (CH-arom.); 132.22, 136.04, 139.37 (C-5, C-1-arom.); 142.35 (CH-8); 151.09 (C-4); 152.50 (CH-2); 157.31 (C-6); 169.23 (C═O); 172.79 (C═N)

IR (CHCl₃); 1743, 1653, 1623, 1597, 1495, 1447, 1333

### Example 13

### Production of 6-[(R,S)-(diphenylmethylideneamino) (ethoxycarbonyl)methyl]-9-(2,3,5-tri-O-acetyl-β-D-ribofuranosyl)purine

The reaction under the same conditions as in Example 11 except that 9-(2,3,5-tri-O-acetyl-β-D-ribofuranosyl)-6-iodopurine (1 mmol) was used instead of 9-benzyl-6-iodopurine (1 mmol), followed by working up gave 6-[(R,S)-(diphenylmethylideneamino)(ethoxycarbonyl)methyl]-9-(2,3,5-tri-O-acetyl-β-D-ribofuranosyl)purine as a yellow amorphous solid (yield 31%).

MS (FAB): 644 (20) [M+H], 386 (32), 312 (35), 139 (100)

HRMS (FAB): for C₃₃H₃₄N₅O₉ [M+H] calculated 644.2357, found 644.2351.

Anal. calculated for C₃₃H₃₃N₅O₉ (643.6): C, 61.58%; H, 5.17%; N, 10.88%; found: C, 61.47%; H, 5.40%; N, 10.50%.

¹H NMR (500 MHz, CDCl₃): 1.18 (t, 3H, J=7.1, CH₃CH₂); 20.9, 2.11, 2.15 (3×s, 3×3H, CH₃CO); 4.22 (d, 2H, J=7.1, CH₃CH₂); 4.37-4.48 (m, 3H, H-4′, H-5′); 5.69 (brm, 1H, H-3′); 5.96-6.00 (m, 2H, H-2′, COCHN); 6.25 (d, 1H, J=5.2, H-1′); 7.26-7.71 (m, 10H, H-arom.); 8.17 (s, 1H, H-8); 8.99 (s, 1H, H-2)

¹³C NMR (100.6 MHz, CDCl₃): 14.05 (CH₃CH₂); 20.36, 20.49, 20.72 (CH₃CO); 61.68 (CH₂CH₃); 63.00, 63.05 (CH₂-5′); 67.74, 67.81 (COCHN); 70.59, 70.62 (CH-3′); 72.96, 73.03 (CH-2′); 80.40 (CH-4′); 86.25, 86.30 (CH-1′); 127.86, 127.94, 128.60, 128.89, 129.24, 130.60 (CH-arom.); 132.94, 135.98, 139.30, (C-5, C-1-arom.); 142.88 (CH-8); 151.41 (C-4); 152.76 (CH-2); 157.85 (C-6); 169.10, 169.30, 169.53, 170.28 (C═O); 173.00 (C═N) IR (CHCl₃); 1749, 1654, 1617, 1595, 1497, 1408, 1370, 1333, 1238

### Example 14

### Production of benzyl (R,S)-3-[9-(tetrahydropyran-2-yl)purin-6-yl]-2-[(tert-butoxycarbonyl)amino]propanoate

The reaction under the same conditions as in Example 1-2 except that 6-iodo-9-(tetrahydropyran-2-yl)purine: (1.5 g, 4.5 mmol) was used instead of 9-benzyl-6-iodopurine (1.35 g, 4.0 mmol) of Example 1-2, followed by working up gave the title compound (1.92 g, yield 88%) as white crystals.

m.p. 101-103° C.

MS (EI): 481 (1, M); 397 (3, M-THP+H); 346 (10, M-COOBn); 262 (17); 218 (10), 206 (28); 188 (20); 162 (100); 134 (54); 91 (66, Bn).

HRMS (EI): for C₂₅H₃₁N₅O₅ calculated 481.2325; found 481.2323.

¹H NMR (500 MHz, CDCl₃): 1.41 (s, 9H, 3×CH₃-Boc); 1.67-1.87 (m, 3H, CH₂ from THP); 2.03-2.15 (m, 3H, CH₂ from THP); 3.62 (dd, 1H, J=4.6 and 16.4, CH_{A}H_{B} from alanyl); 3.79 (m, 1H, H-5′); 3.90 (dd, 1H, J=5.5 and 16.4, CH_{A}H_{B} from alanyl); 4.19 (m, 1H, H-5′); 4.97 (m, 1H, CHNH); 5.10 (m, 2H, CH₂Ph); 5.77. (d, 1H, J=10.5, H-1′); 6.10 (m, 1H, NH); 7.24 (m, 5H, arom.); 8.22 (s, 1H, H-8); 8.77 (s, 1H, H-2).

¹³C NMR (125.8 MHz, CDCl₃): 22.74 (CH₂-THP); 24.84 (CH₂-THP); 28.29 (CH₃-tBu); 31.79 (CH₂-THP); 34.53 (CH₂ from alanyl); 51.79 (CH from alanyl); 66.99 (CH₂Ph); 68.83 (CH₂-5′); 79.75 (C-tBu); 81.98 (CH-1′); 128.07, 128.10 and 128.32 (3×CH-arom.) 132.95 (C-5); 135.49 (C-arom.); 141.92 (CH-8); 149.98 (C-4); 152.05 (CH-2); 155.54 (CO-Boc); 157.80 (C-6); 171.60 (COOBn). IR (CHCl₃): 3436, 2983, 2867, 1735, 1710, 1599, 1584, 1498, 1456, 1369, 1335, 1250, 1163, 1086, 1046, 913.

Anal. calculated for C₂₃H₃₁N₅O₅ (481.5): C, 62.36%; H, 6.49%; N, 14.54%; found: C, 62.04%; H, 6.79%; N, 14.13%.

### Example 15

### Production of benzyl (R,S)-3-[9-(2,3,5-tri-O-acetyl-β-D-ribofuranosyl)purin-6-yl]-2-[(tert-butoxycarbonyl)amino]propanoate

The reaction under the same conditions as in Example 1-2 except that 6-iodo-9-(2,3,5-tri-O-acetyl-β-D-ribofuranosyl)purine (2.02 g, 4 mmol) was used instead of 9-benzyl-6-iodopurine (1.35 g, 4.0 nmol) of Example 1-2, followed by working up gave the title compound (1.96 g, yield 75%) as a yellow amorphous solid.

MS (FAB): 656 (47, M+1); 600 (25); 342 (100); 298 (35); 281 (66).

HRMS (FAB): for. C₃₁H₃₈N₅O₁₁ calculated 656.2568; found 656.2549.

¹H NMR (500 MHz, CDCl₃): 1.43 (s, 9H, 3×CH₃-Boc); 2.10 (s, 3H, CH₃); 2,14 (s, 3H, CH₃); 2.18 (s, 3H, CH₃); 3.65 (ddd, 1H, J=4.6, 13.4 and 15.9, CH_{A}H_{B} from alanyl); 3.90 (td, 1H, J=6.0 and 16.0, CH_{A}H_{B} from alanyl); 4.38-4.51 (m, 3H, H-4′+H-5′) 5.00 (m, 1H, CH from alanyl); 5.07-5.17 (m, 2H, CH₂Ph); 5.69 (dd, 1H, J=4.5 and 9.7, H-3′); 5.97 (m, 1H, H-2′); 6.07 (br t, 1H, J=7.2, NH); 6.24 (t, 1H, J=5.4, H-1′); 7.26 (m, 5H, arom.); 8.177 and 8.184 (2×s, 1H, H-8); 8.78 and 8.79 (2×s, 1H, H-2).

¹³C NMR (125.8 MHz, CDCl₃): 20.38, 20.48 and 20.70 (3×CH₃CO) 28.23 (CH₃. tBu); 34.47 and 34.59 (CH₂ from alanyl); 51.61 and 51.66 (CH from alanyl); 62.98 (CH₂-5′); 67.03 (CH₂Ph); 70.53 (CH-3′); 72.92 and 73.05 (CH-2′); 79.80 (C-tBu); 80.32 (CH-4′); 86.33 and 86.41 (CH-1′); 128.12 and 128.30 (CH-arom.); 133.42 and 133.48 (C-5); 135.35 (C-arom.); 142.42 and 142.53 (CH-8); 150.21 and 150.24 (C-4); 152.25 (CH-2); 155.48 (CO, Boc); 158.32 (C-6); 169.28, 169.51 and 170.24 (3×COCH₃); 171.48 (COOBn).

IR (CDCl₃): 3436, 3029, 3011, 2983, 1749, 1711, 1599, 1498, 1456, 1399, 1336, 1235, 1205, 1163, 1097, 1050, 911, 645, 698.

Anal. calculated for C₃₁H₃₇N₅O₁₁ (655.6): C, 56.79%; H, 5.69%; N, 10.68%; found: C, 57.06%; H, 6.04%; N, 10.22%.

### Reference Example 1

### 9-(3,5-di-O-β-toluyl-β-D-2′-deoxyribofuranosyl)-6-iodopurine white crystals, m.p. 139-140° C.

MS (FAB): 599 (6, M+1); 353 (6); 247 (19); 185 (10); 119 (82); 91 (38); 81 (100).

HRMS (FAB): for C₂₆H₂₄₁N₄O₅ calculated 599.0791; found 599.0787.

¹H NMR (500 MHz, CDCl₃): 2.41 (s, 3H, CH₃); 2.45 (s, 3H, CH₃) 2.89 (ddd, 1H, J=2.2, 5,9 and 14.2, H-2′a); 3.19 (ddd, 1H, J=6.4, 7.9 and 14.3, H-2′b); 4.66 (m, 2H, H-4′+H-5′a); 4.79 (m, 1H, H-5′); 5.84 (m, 1H, H-3′); 6.55 (dd, 1H, J=5.9 and 8.1, H-1′); 7.2.1 (d, 2H, J=8.0, arom.); 7.28 (d, 2H, J=8.0, arom.); 7.85 (d, 2H, J=8.2, arom.); 7.97 (d, 2H, J 8.2, arom.); 8.30 (s, 1H, H-8); 8.55 (s, 1H, H-2).

¹³C NMR (100 MHz, CDCl₃): 21.69 (CH₃); 21.72 (CH₃); 37.90 (CH₂-2′); 63,74 (CH₂-5′); 74.99 (CH-3′); 83.41 (CH-4′); 85.44 (CH-1′); 122.42 (C-6); 126.30 and 126.48 (2×C-1-arom.); 129.29, 129.55 and 129.80 (3×CH-arom.); 139.22 (C-5); 142.50 (CH-8); 144.26 and 144.61 (2×C-4-arom.); 147.33 (C-4); 151.94 (CH-2); 165.90 and 166.05 (2×CO).

IR (CDCl₃): 3033, 3003, 1721, 1612, 1581, 1554, 1485, 1429, 1333, 1269, 1178, 1102, 1021, 914, 839, 691.

Anal. calculated for C₂₆H₂₃₁N₄O₅ (598.4): C, 52.19%; H, 3.87%, I 21.21%; N, 9.36%; found: C, 52.27%; H, 4.00%; I, 21.17%; N, 9.26%.

### Example 16

### Production of benzyl (R,S)-3-[9-(3,5-di-O-p-toluyl-β-D-2′-deoxyribofuranosyl)purin-6-yl]-2-[(tert-butoxycarbonyl) amino]propanoate

The reaction under the same conditions as in Example 1-2 except that 9-(3,5-di-O-p-toluyl-β-D-2′-deoxyribofuranosyl)-6-iodopurine (1.88 g, 3.14 mmol) in Reference Example 1 was used instead of 9-benzyl-6-iodopurine (1.35 g, 4.0 mmol) of Example 1-2, followed by working up gave the title compound (2.22 g, yield 94%) as a yellow amorphous solid.

MS (FAB): 750 (10, M+1); 398 (18); 342 (100); 321 (56); 298 (36); 281 (66); 252 (25).

HRMS (FAB): for C₄₁H₄₃N₅O₉ calculated 750.3139; found 750.3140.

¹H NMR (500 MHz, CDCl₃): 1.43 (s, 9H, tBu); 2.43 and 2.47 (2×s, 6H, 2×CH₃ from toluyl); 2.83 (m, 1H, H-2′); 3.15 (m, 1H, H-2′); 3.59 (m, 1H, CH_{A}H_{B} from alanyl); 3.87 (dt, 1H, J=5.5 and 14.7, CH_{A}H_{B} from alanyl); 4.65-4.70 (m, 2H, H-4′+H-5′); 4.75-4.80 (m, 1H, H-5′); 4.99 (m, 1H, CH from alanyl); 5.10 (m, 2H, CH₂Bn); 5.85 (m, 1H, H-3′); 6.08 (2×d, 1H, J=8.7, NH); 6.59 (dd, 1H, J=6.0 and 8.0); 7.21-7.32 (m, 9H, arom.); 7.94 (dd, 2H, J=3.7 and 8.2, arom.); 8.00 (d, 2H, J=8.2, arom.); 8.20 (s, 1H, H-8); 8.74 and 8.75 (2×s, 1H, H-2).

¹³C NMR (125.8 MHz, CDCl₃): 21.69 and 21.75 (2×CH₃ from toluyl); 28.29 (C(CH₃)₃); 34.52 and 34.63 (CH₂ from alanyl); 37.76 and 37.84 (CH₂-2′); 51.74 (CH from alanyl); 63.97 (CH₂-5′); 67.03 and 67.05 (CH₂Ph); 75.06 (CH-3′); 79.81 (CC(CH₃)₃); 83.08 and 83.11 (CH-4′); 84.79 (CH-1′); 126.35 and 126.62 (2×C-p-arom.); 128.13, 128.33, 129.31, 129.64 and 129.82 (5×CH-arom.); 133.47 (C-5); 135.40 (C-i-arom.); 142.32 (CH-8); 144.23 and 144.59 (2×C-i-arom.); 150.21 (C-4); 152.09 (CH-2); 155.55 (CO from Boc); 158.10 (C-6); 165.94 and 166.16 (CO from toluyl); 171.52 (COOBn).

IR (CDCl₃): 3435, 3020, 2983, 1719, 1612, 1599, 1498, 1456, 1369, 1335, 1269, 1179, 1163, 1102, 1021.

Anal. calculated for C₄₁H₄₃N₅O₉ (749.8): C, 65.68%; H, 5.78%; N, 9.34%; found: C, 65.80%; H, 6.06%; N, 8.93%.

### Example 17

### Production of benzyl (R,S)-3-[9-(2,3,5-tri-O-acetyl-β-D-ribofuranosyl)purin-6-yl]-2-[(benzyloxycarbonyl)amino]propanoate

The reaction in the same manner as in Example 1-2 gave the title compound.

¹H NMR (500 MHz, CDCl₃): 2.08 (s, 3H, CH₃); 2,11 (s, 3H, CH₃); 2.16 (s, 3H, CH₃); 3.66 (td, 1H, J=4.4 and 15.6, CH_{A}H_{B} from alanyl); 3.93 (td, 1H, J=5.6 and 16.0, CH_{A}H_{B} from alanyl); 4.36-4.48. (m, 3H, H-4′+H-5′); 5.05 (m, 1H, CH from alanyl); 5.10 (s, 2H, CH₂Ph); 5.12 (s, 2H, CH₂Ph); 5.66 (m, 1H, H-3′); 5.94 (dt, 1H, J=5.4 and 17.5H-2′); 6.21 (t, 1H, J=5.5, H-1′); 6.45 (m, 1H, NH); 7.20-7.34 (m, 10H, arom.); 8.166 and 8.173 (2×s, 1H, H-8); 8.72 and 8.74 (2×s, 1H, H-2).

¹³C NMR (125.8, MHz, CDCl₃): 20.32, 20.46 and 20.67 (3×CH₃CO); 34.30 and 34.42 (CH₂ from alanyl); 52.12 (CH from alanyl); 62.98 (CH₂-5′); 66.93 and 67.03 (2×CH₂Ph); 70.55 (CH-3′); 72.98 and 73.11 (CH-2′); 80.38 (CH-4′); 86.38 and 86.46 (CH-1′); 128.04, 128.18, 128.36 and 128.43 (CH-arom.); 133.23 and 133.29 (C-5); 135.31 and 136.31 (2×C-arom.); 142.55 and 142.66 (CH-8); 150.30 (C-4); 152.25 (CH-2); 156.10 (NCO); 158.07 (C-6); 169.26, 169.49 and 170.23 (3×COCH₃); 171.08 (COOBn).

IR (CDCl₃): 3430, 3030, 3013, 1750, 1599, 1500, 1456, 1410, 1375, 1336, 1229, 909, 645, 603.

### Example 18

### Production of benzyl (R,S)-3-[9-(3,5-di-O-p-toluyl-β-D-2′-deoxyribofuranosyl)purin-6-yl]-2-[(benzyloxycarbonyl)amino]propanoate

The reaction in the same manner as in Example 1-2 gave the title compound as a yellowish amorphous solid.

MS (FAB): 784 (8, M+1); 432 (23); 321 (8); 281 (11); 154 (21); 119 (82); 91 (100%).

HRMS (FAB): for C₄₄H₄₂N₅O₉ calculated 784.2983; found 784.2955.

¹H NMR (500 MHz, CDCl₃): 2.40 (s, 3H, CH₃); 2.45 (s, 3H, CH₃) 2.83 (dm, 1H, J=14.2, H-2′); 3.15 (m, 1H, H-2′); 3.63 (ddd, 1H, J=4.4, 16.0 and 25.2, CH_{A}H_{B} from alanyl); 3.93 (ddd, 1H, J=5.6, 14.0 and 15.8, CH_{A}H_{B} from alanyl); 4.67 (m, 1H, H-4′); 4.68 (m, 1H, H-5′); 4.77 (m, 1H, H-5′); 5;06 (m, 1H, CH from alanyl); 5.10 (s, 1H, CH₂Ph); 5.11 (s, 1H, CH₂Ph); 5.83 (br d, 1H, J=4.7, H-3′); 6.49 (dd, 1H, J=8.8 and 15.4, NH); 6.56 (t, 1H, J=6.6, H-1′); 7.18-7.34 (m, 14H, arom.); 7.92 (dd, 2H, J=4.7 and 7.8, arom.); 7.99 (d, 2H, J=8.1, arom.); 8.18 (s, 1H, H-8); 8.69 and 8.70 (2×s, 1H, H-2).

¹³C NMR (125.8 MHz, CDCl₃): 21.62 and 21.70 (2×CH₃); 34.21 and 34.34 (CH₂ from alanyl); 37.66 and 37.75 (CH₂-2′); 51.12 (CH from alanyl); 63.91 (CH₂-5′); 66.88 and 67.08 (2×CH₂Ph); 74.99° (CH-3′); 83.03 and 83.06 (CH-4′); 84.76 and 84.79 (CH-17); 126.31 and 126.58 (2×C-p-arom.); 128.03, 128.14, 128.30, 128.32, 128.42, 129.26, 129.59 and 129.78 (8×CH-arom.); 133.38 and 133.43 (C-5); 135.27 and 136.28 (2×C-i-arom.); 142.39 and 142.42 (CH-8); 144.16 and 144.53 (2×C-i-arom.); 150.18 and 150.22 (C-4); 151.99 (CH-2); 156.07 and 156.09 (NCO); 157.75 (C-6); 165.88 and 166.10 (CO from toluyl); 171.10 (COOBn).

IR (CDCl₃): 3429, 3032, 3013, 1721, 1612, 1599, 1500, 1456, 1408, 1387, 1335, 1269, 1102, 1021, 938, 841, 646.

Anal. calculated for C₄₄H₄₁N₅O₉ (783.8): C, 67.42%; H, 5.27%; N, 8.93%; found: C, 67.07%; H, 5.46%; N, 8.71%.

### Example 19

### Production of (R,S)-3-(9-Benzylpurin-6-yl)-2-[(tert-butoxycarbonyl)amino]propanoic acid

Benzyl (R,S)-3-(9-benzylpurin-6-yl)-2-[(tert-butoxycarbonyl)amino]propanoate (1.9 g, 3.9 mmol) obtained in Example 1-2 in ethanol (140 ml) was hydrogenated under slight overpressure in the presence of Pd/C catalyst (10 wt %, 180 mg) for 6 hr (the progress was monitored by TLC). The catalyst was filtered off through Celite pad and the filtrate was evaporated in vacuo and crystallized from ethanol to give the title compound (1.38 g, yield 89%) as white crystals.

m.p. 172-175° C.

MS (FAB): 398 (16, M+1); 342 (19); 281 (7, M-NHBoc); 252 (19); 225 (23); 135 (6); 91 (100, Bn).

HRMS (FAB): for C₂₀H₂₃N₅O₄ calculated 398.1828, found 398.1840.

¹H NMR (200 MHz, DMSO-d): 1.28 (s, 9H, 3×CH₃-Boc); 3.42 (dd, 1H, J=7.8 and 14.9, CH_{A}H_{B}CH); 3.57 (dd, 1H, J=6.9 and 14.9, CH_{A}H_{B}CH); 4.75 (m, 1H, CHCH₂); 5.50 (s, 2H, CH₂Ph); 7.20-7.33 (m, 5H, arom.); 8.73 (s, 1H, H-8); 8.86 (s, 1H, H-2).

¹³C NMR (50.3 MHz, DMSO-d₆): 28.29 (CH₃); 34.48 (CH₂CH); 46.71 (CH₂Ph); 52.00 (CHCH₂); 78.32 (C(CH₃)); 127.89, 128.18 and 128.98 (3×CH-arom.); 132.51 (C-5); 136.71 (C-arom.); 146.20 (CH-8); 150.72 (C-4); 151.67 (CH-2); 155.40 (COO-tBu); 157.73° (C-6); 173.27 (COOH).

IR (KBr): 3210, 2980, 2932, 1726, 1701, 1653, 1599, 1532, 1504, 1455, 1406, 1368, 1335, 1161, 1050.

Anal. calculated for C₂₀H₂₃N₅O₄ (397.4): C, 60.44%; H, 5.83%; N, 17.62%; found: C, 60.22%; H, 5.92%; N, 17.36%.

### Example 20

### Production of (R,S)-2-tert-butoxycarbonylamino-3-(9H-purin-6-yl)propionic acid

Benzyl (R,S)-3-[9-(tetrahydropyran-2-yl)purin-6-yl]-2-[(tert-butoxycarbonyl)amino]propanoate (867 mg, 1.8 mmol) obtained in Example 14 in ethanol (120 ml) was hydrogenated under slight overpressure in the presence of Pd/C catalyst (10 wt %, 90 mg) for 6 hr. The catalyst was filtered off through Celite pad and the filtrate was evaporated in vacuo and crystallized from methanol/ethyl acetate to give the title compound (465 mg, yield 84%) as white crystals.

m.p. 186-189° C. with decomposition.

MS (FAB) 308 (21, M+1); 252 (50); 208 (23, M-Boc+2H); 191 (35); 162 (47); 135 (76)

HRMS (FAB): for C₁₃H₁₈N₅O₄ calculated 308.1359; found 308.1352.

¹H NMR (500 MHz, DMSO-d₆): 1.30 (s, 9H, 3×CH₃ from tBu); 3.40 (dd, 1H, J=8.1 and 14.5, CH_{A}H_{B}); 3.50 (dd, 1H, J=3.5 and 14.5, CH_{A}CH_{B}); 4.70 (br, 1H, CH from alanyl); 7.19 (d, 1H, J 8.3, NH); 8.55 (br s, 1H, H-8); 8.79 (s, 1H, H-2); 13.48 (v br, 1H, COOH).

¹³C NMR (125.8 MHz, DMSO-d₆): 28.07 (CH₃); 34.62 (CH₂ from alanyl); 51.81 (CH from alanyl); 78.09 (C from tu); 151.29 (C-4, HMBC experiment); 151.55 (CH-2); 155.17 (CO-tBu); 157.08 (C-6, HMBC experiment); 173.13 (COOH).

IR (KBr): 3257, 3978, 2815, 2557, 1928, 1735, 1711, 1625, 1573, 1532, 1447, 1383, 1328, 1298, 1250, 1164, 1045, 809, 640.

Anal. calculated for C₁₃H₁₇N₅O₄ (307.3): C, 50.81%; H, 5.58%; N, 22.79%; found: C, 50.77%; H, 6.05%; N, 22.35%;

### Example 21

### Production of (R,S)-2-amino-3-(9-benzylpurin-6-yl)propanoic acid trifluoracetate

TFA (0.8 ml) was added at 0° C. to (R,S)-3-(9-benzylpurin-6-yl)-2-[(tert-butoxycarbonyl)amino]propanoic acid (120 mg, 0.3 mmol) obtained in Example 19 in CH₂Cl₂ (8 ml). The reaction mixture was stirred at ambient temperature for 1 hr. Then the solvent was evaporated in vacuo and the residue was codestileted with CH₂Cl₂. The crude product was recrystallized from ethyl acetate/methanol to give the title compound (95 mg, yield 72%) as white crystals.

m.p. 243-244° C.

MS (FAB): 298-(100, M+1); 252 (17, M−COOH); 225 (33); 157 (12); 135 (10); 91 (98, Bn).

HRMS (FAB): for C₁₅H₁₆N₅O₂ calculated 298.1304; found 298.1307.

¹H NMR (200 MHz, DMSO-d₆): 3.61 (dd, 1H, J=6.9 and 16.4, CH_{A}H_{B}CH); 3.75 (dd, 1H, J=5.5 and 16.4, CH_{A}H_{B}CH); 4.64 (t, 1H, J=5.7, CHCH₂); 5.52 (s, 2H, CH₂Ph); 7.29-7.38 (m, 5H, arom.); 8.44 (v br, NH₃⁺); 8.78 (s, 1H, H-8); 8.87 (s, 1H, H-2).

¹³C NMR (50.3 MHz, DMSO-d₆): 32.44 (CH₂CH); 46.80 ( CH₂Ph); 50.35 (CHCH₂); 127.96, 128.24 and 129.02 (3×CH-arom.); 132.41 (C-5); 136.68 (C-arom.); 146.50 (CH-8); 150.84 (C-4); 151.93 (CH-2); 155.49 (C-6); 170.43 (CHCOO).

IR (KBr): 1732, 1692, 1603, 1530, 1506, 1406, 1335, 1264, 1197, 1183, 1131.

Anal. calculated for C₁₇H₁₆F₃Nr₅O₄ (411.3): C, 49.64%; H, 3.92%; N, 17.03%; found C, 49.38%; H, 3.97%; N, 16.77%.

### Example 22

### Production of (R,S)-2-amino-3-(9-benzylpurin-6-yl)propanoic acid hydrochloride

(R,S)-3-(9-Benzylpurin-6-yl)-2-[(tert-butoxycarbonyl)amino]propanoic acid (398 mg, 1 mmol) obtained in Example 19 in ethyl acetate saturated with hydrochloric acid (aprox. 1.7 M) was stirred at ambient temperature for 8 hr, then the precipitate was filtered and recrystallized from ethanol to give the title compound (280 mg, yield 84%) as white crystals.

m.p. 231-235° C.

¹H NMR (200 MHz, DMSO-d₆): 3.68 (dd, 1H, J=6.4 and 16.4, CH_{A}H_{B}CH); 3.80 (dd, 1H, J=6.2, and 16.4, CH_{A}H_{B}CH); 4.64 (br s, 1H, CHCH₂); 5.53 (s, 2H, CH₂Ph); 7.27-7.39 (m, 5H, arom.); 8.68 (br s, 3H, NH₃⁺); 8.81 (s, 1H, H-8); 8.86 (s, 1H, H-2). ¹³C NMR (50.3 MHz, DMSO-d₆): 32.40 (CH₂CH); 46.79 (CH₂Ph); 50.18 (CHCH₂); 127.96, 128.23 and 129.02 (3×CH arom.); 132.36 (C-5); 136.71 (C arom.); 146.51 (CH-8); 150.82 (C-4); 151.90 (CH-2); 155.60 (C-6); 170.31 (COO).

Anal. calculated for C₁₅H₁₆ClN₅O₂ (333.8): C, 53.98%; H, 4.83%; N, 20.98%; Cl, 10.62%; found: C, 53.79%; H, 4.91%; N, 20.22%; Cl, 10.85%.

### Example 23

### Production of (R,S)-2-amino-3-(9H-purin-6-yl)propanoic acid trifluoracetate

The reaction in the same manner as in Example 21 gave the title compound (90 mg, yield 86%) as white crystals.

m.p. 163-166° C.

MS (FAB): 208 (100, M+1); 181 (36); 162 (33); 149 (18); 135 (65); 133 (45); 36 (110).

MS (FAB): for C₈H₁₀N₅O₂ calculated 208.0835; found 208.0848.

¹H NMR (500 MHz, DMSO-d₆): 3.62. (dd, 1H, J=6.9 and 16.5, CH_{A}H_{B}); 3.73 (dd, 1H, J=5.3 and 16.5, CH_{A}H_{B}); 4.64 (dd, 1H, J. 5.3 and 6.4, HCH₂); 8.46 (v br, NH₃⁺); 8.60 (s, 1H, H-8) 8.83 (s, 1H, H-2).

¹³C NMR (125.7 MHz, DMSO-d₆): 32.35 (CH₂); 50.09 (CH from alanyl); 145.20 (CH-8); 151.51 (CH-2); 153.33 (C-6, HMBC experiment); 170.22 (CO).

IR (KBr): 3424, 3183, 3159, 2853, 2698, 2632, 1698, 1685, 1608, 1517, 1491, 1425, 1399, 1338, 1264, 1224, 1196, 1133, 836, 796, 723, 637.

Anal. calculated for C₁₀H₁₀F₃N₅O₄ (321.2): C, 37.39%; H, 3.14%; N, 21.80%; found C, 37.44%; H, 3.22%; N, 21.41%.

### Example 24

### Production of (R,S)-2-amino-3-(9H-purin-6-yl)propanoic acid dihydrochloride

The reaction in the same manner as in Example 22 gave the title compound (yield 99%) as white crystals.

m.p. more than 280° C.

¹H NMR (200 MHz, DMSO-d₆): 3.74 (dd, 1H, J=6.4 and 16.4, CH_{A}H_{B}); 3.85 (dd, 1H, J=5.9 and 16.4, CH_{A}H_{B}); 4.69 (br s, 1H, CHCH₂); 5.90 (v br, NH₂⁺); 8.71 (br s; NH₃⁺); 8.88 (s, 1H, H-8) 8.94 (s, 1H, H-2).

¹³C NMR (50.3 MHz, DMSO-d₆): 32.40 (CH₂); 50.12 (CH from alanyl); 128.79 (C-5); 146.11 (CH-8); 151.29 (CH-2); 153.01 and 153.67 (C-6 and C-4); 170.14 (CO).

### Example 25

### Production of (R,S)-2-amino-3-[9-(2,3,5-tri-O-acetyl-A-D-ribofuranosyl)purin-6-yl]propionic acid benzyl ester trifluoracetate

TFA (1.8 ml, 2.3 mmol) was slowly added at 0° C. to benzyl (R,S)-3-[9-(2,3,5-tri-O-acetyl-β-D-ribofuranosyl)purin-6-yl]-2-[(tert-butoxycarbonyl)amino]propanoate (1.4 g, 2.1 mmol) obtained in Example 15 in CH₂Cl₂ (30 ml). The reaction mixture was stirred at room temperature overnight. Then the solvent was evaporated in vacuo and the residue was codestileted with CH₂Cl₂. The crude product was chromatographed on a silica gel column (methanol/chloroform, 1:9) to give the title compound (1.4 g, yield 97%) as a yellowish amorphous solid.

MS (FAB): 556 (14, M+1); 298 (26); 259 (25); 162 (33); 139 (100); 135 (57); 97 (74); 91 (98, Bn).

HRMS (FAB): for C₂₆H₃₀N₅O₉ calculated 556.2044, found 556.2057.

¹H NMR (500 MHz, CDCl₃): 2.06 (d, 3H, J=2.08, CH₃CO); 2.10 (s, 3H, CH₃CO); 2.15 (d, 3H, J=2.23, CH₃CO); 3.88-4.04 (m, 2H, CH₂ from alanyl); 4.39-4.49 (m, 3H, H-4′+H-5′); 4.70 (br s, 1H, CH form alanyl); 5.04 (dd, 1H, J=12.3 and 15.1, CH_{A}H_{B}Ph); 5.16 (dd, 1H, J=9.0 and 11.6, CH_{A}H_{B}Ph); 5.64. (dd, 1H, J=4.5 and 9.5, H-3′); 5.91 (2×t, 1H, J=5.5, H-2′); 6.21 (2×d, 1H, J=5.2, H-1′); 7.09-7.25 (m, 5H, arom.); 8.25 (s, 1H, H-8); 8.67 and 8.69 (2×s, 1H, H-2).

¹³C NMR (125.8 MHz, CDCl₃): 20.22, 20.45 and 20.65 (3×CH₃) 30.91 (CH₂ from alanyl); 51.13 and 51.17 (CH from alanyl); 63.03 and 63.07 (CH₂-5′); 68.42 and 68.46 (H₂Ph); 70.57 and 70.63 (CH-3′); 72.90 and 73.15 (CH-2′); 80.51 and 80.57 (CH-4′); 86.30 and 86.58 (CH-1′); 128.37, 128.41, 128.52, 128.55 and 128.65 (5×CH-arom.); 132.32 and 132.40 (C-5); 134.08 and 134.11 (C-arom.); 143.32 (CH-8); 150.43 (C-4); 151.98 (CH-2); 156.15 (C-6); 168.19 (COOBn); 169.53, 169.66 and 170.51 (3×COCH₃).

IR (CHCl₃): 3031, 3009, 1751, 1680, 1603, 1499, 1457, 1374, 1338, 1226, 1206, 1143, 801, 644.

### Example 26

### Production of methyl (R,S)-3-{4-[9-(tetrahydropyran-2-yl)purin-6-yl]phenyl}-2-[(tert-butoxycarbonyl)amino]propanoate

The reaction under the same conditions as in Example 2-2 except that 6-iodo-9-(tetrahydropyran-2-yl)purine (1.33 g, 3 mmol) and methyl (R,S)-2-[(tert-butoxycarbonyl) amino]-3-[4-(trimethylstannanyl)phenyl]propionate (878 mg, 2.66 mmol) were used instead of 9-benzyl-6-iodopurine (547 mg, 1.65 mmol) and methyl (R,S)-2-[(tert-butoxycarbonyl)amino]-3-[4-(trimethylstannanyl)phenyl]propionate (827 mg, 1.87 mmol) of Example 2-2, followed by working up gave the title compound (687 mg, yield 53%) as white crystals.

m.p. 144-147° C.

MS (EI): 481 (0.5, M); 364 (4); 341 (6); 324 (7); 294 (15); 280 (13); 238 (11); 210 (100).

HRMS (EI): for C₂₅H₃₁N₅O₃ calculated 481.2325, found 481.2302.

¹H NMR (200 MHz, CDCl₃): 1.43 (s, 9H, (CH₃)₃); 1.65-1.87 (m, 3 Hi CH₂ from THP); 2.02-2.22° (m, 3H, CH₂ from THP); 3.20 (br d, 2H, J=4.8, CH₂ from alanyl); 3.73 (s, 3H, OCH₃); 3.85 (dd, 1H, J=3.0 and 11.4, H-5′a), 4.21 (dt, 1H, J=2.0 and 11.4, H-5′b); 4.66 (dd, 1H, J=5.9 and 13.8, CHNH); 5.05 (d, 1H, J=7.9, NH); 5.86 (dd, 1H, J=3.4 and 9.5, H-1′); 7.33 (d, 2H, J=8.3, arom.); 8.34 (s, 1H, H-8); 8.73 (d, 2H, J=8.3, arom.); 9.01 (s, 1H, H-2).

¹³C NMR (50.3 MHz, CDCl₃): *22.77 and 24.83 (2×CH₂ THP); 28.26 ((CH₃)₃); 31.81 (CH₂ THP); 38.12 (CH₂ from alanyl); 52.27 (OCH₃); 54.28 (CHNH); 68.87 (CH₂-5′); 79.98 (C(CH₃)₃); 81.92 (CH-1′); 129.65 and 129.93 (2×CH-arom.); 130.97 (C-5); 134.46 (C-1-arom.); 139.19 (C-p-arom.); 141.98 (CH-8); 151.64 (C-4); 152.37 (CH-2); 154.52 (C-6); 155.04 (COO from Boc); 172.09 (COOMe).

IR (CHCl₃): 3436, 2983, 1743, 1710, 1584, 1560, 1499, 1449, 1166.

Anal. calculated for C₂₅H₃₁N₅O₅ (481.5): C, 62.36%; H, 6.49%; N, 14.54%; found: C, 62.37%; H, 6.72%; N, 14.14%.

### Example 27

### Production of methyl (R,S)-3-{4-[9-(2,3,5-tri-O-acetyl-p-D-ribofuranosyl)purin-6-yl]phenyl}-2-[(tert-butoxycarbonyl) amino]propanoate

The reaction under the same conditions as in Example 2-2 except that 6-iodo-9-(2,3,5-tri-O-acetyl-β-D-ribofuranosyl)purine (1.33 g, 3 mmol) and methyl (R,S)-2-[(tert-butoxycarbonyl)amino]-3-[4-(trimethylstannanyl)phenyl]propionate. (1.34 g, 2.66 mmol) were used instead of 9-benzyl-6-iodopurine (547 mg, 1.65 mmol) and methyl (R,S)-2-[(tert-butoxycarbonyl)amino]-3-[4-(trimethylstannanyl)phenyl]propionate (827 mg, 1.87 mmol) of Example 2-2, followed by working up gave the title compound (961 mg, yield 55%) as a colorless amorphous solid.

MS (FAB): 656 (3, M+H); 600 (3); 342 (34); 259 (19); 238 (22); 210 (38); 139 (92); 97 (79).

HRMS (FAB): for C₃₁H₃₇N₅O₁₁ calculated 656.2568, found 656.2525.

¹H NMR (200 MHz, CDCl₃): 1.43 (s, 9H, (CH₃)₃); 2.10, 2.15 and 2.17 (3×s, 3×3H, 3×CH₃ from Ac); 3.19 (m, 2H, CH₂); 3.73 (s, 3H, OCH₃); 4.35-4.53 (m, 2H, H-5′ and H-4′); 4.66 (dd, 1H, J=5.4 and 13.7, CHCH₂); 5.03 (d, 1H, J=8.1, NH); 5.72 (dd, 1H, J=4.4 and 5.4, H-3′); 6.02 (t, 1H, J=5.4, H-2′); 6.30 (d, 1H, J=5.4, H-1′); 7.33 (d, 2H, J=8.3, arom.); 8.28 (s, 1H, H-8); 8.71 (d, 2H, J=8.3, arom.); 9.02 (s, 1H′, H-2).

¹³C NMR (125.8 MHz, CDCl₃): 20.34, 20.49 and 20.71 (3×H₃CO); 28.27 (CH₃ from tBu); 38.15 (CH₂CHNH); 52.24 (OCH₃); 54.25 (CHNH); 63.01 (CH2-5′); 70.58 (CH-3′); 73.03 (CH-2′); 79.97 (C(CH₃)₃); 80.33 (CH-4′); 86.36 (CH-1′); 129.67 and 129.94 (CH-arom.); 131.48 (C-arom.); 134.15 (C-5); 139.47 (C-arom.); 142.48 (CH-8); 151.95 (C-4); 152.61 (CH-2); 155.00 (C-6); 169.33, 169.54 and 170.27 (3×CO from Ac); 172.06 (COOMe).

IR (CHCl₃): 3438, 3010, 2984, 1749, 1711, 1673, 1584, 1498, 1369, 1234, 1167, 1062, 925, 866, 804, 667, 603.

### Example 28

### Production of (R,S)-3-[4-(9-benzylpurin-6-yl)phenyl]-2-[(tert-butoxycarbonyl) amino]propanoic acid

Methyl (R,S)-3-[4-(9-benzylpurin-6-yl)phenyl]-2-[(tert-butoxycarbonyl)amino]propanoate (245 mg, 0.5 mmol) obtained in Example 2-2 was dissolved in THF (17 ml) and 0.2 M aqueous solution of NaOH (4.5 ml) was added. The reaction mixture was stirred at room temperature for 1.5 hr. Then 1% aqueous HCl was added to adjust to pH 4, and the mixture was diluted with water (50 ml) and washed with ethyl acetate (80 ml). The organic extract was evaporated and recrystallized from ethyl acetate to give the title compound (220 mg, yield 93%) as white crystals.

m.p. 214-216° C.

MS (FAB): 474 (14, M+1); 418 (30); 328 (8), 300 (11, M-CHCOOH(NHBoc)+1); 91 (100, Bn).

HRMS (FAB): for C₂₆H₂₈N₅O₄ calculated 474.2141; found 474.2139.

¹ NMR (200 MHz, DMSO-d₆): 1.31 (s, 9H, 3×CH₃-Boc); 2.93 (dd, 1H, J=10.4 and 13.8, CH_{A}H_{B}CH); 3.13 (dd, 1H, J=4.5 and 13.8, CH_{A}H_{B}CH); 4.18 (m, 1H, CHNH); 5.55 (s, 2H, CH₂Ph); 7.22 (d, 1H, J=8.3, arom.); 7.28-7.40 (m, 4H, arom.); 7.47 (d, 2H, J=8.2, arom.); 8.75 (d, 2H, J=8.2, arom.); 8.82 (s, 1H, H-8); 8.97 (s, 1H, H-2); 12.70 (very br, 1H, COOH).

¹³C NMR (50.3 MHz, DMSO-d₆): 28.34 (CH₃); 36.63 (CH₂CH); 46.71 (CH₂Ph); 55.14 (CH₂CH); 78.31 (C(CH₃)₃); 127.88, 128.16, 128.99, 129.42 and 129.68 (5×CH-arom.); 130.38 (C-5); 133.76, 136.74 and 141.70 (3×C-arom.); 146.61 (CH-8); 152.20 (CH-2); 152.46, 152.88 and 155.69 (C-4, C-6 and COOtBu); 173.71 (COOH).

IR (KBr): 3393, 2979, 2932, 1707, 1585, 1560, 1509, 1457, 1367, 1328, 1243, 1187, 1165, 1055, 727.

Anal. calculated for C₂₆H₂₇N₅O₄ (473.5): C, 65.95%; H, 5.75%; N, 14.79%; found: C, 65.72%; 5.83%; 14.65%.

### Example 29

### Production of (R,S)-2-amino-3-[4-(9-benzylpurin-6-yl)phenyl]propionic acid trifluoracetate

(R,S)-3-[4-(9-Benzylpurin-6-yl) phenyl]-2-[(tert-butoxycarbonyl)amino]propanoic acid (115 mg, 0.24 mmol) obtained in Example 28 was dissolved in CH₂Cl₂ (9 ml) and TFA (1 ml) was added at 0° C. The reaction mixture was stirred at room temperature for 5 hr. Then the solvent was evaporated in vacuo and the residue was codestileted with CH₂Cl₂. The crude product was recrystallized from ethyl acetate/methanol to give the title compound (89 mg, yield 77%) as white crystals.

m.p. 194-197° C.

MS (FAB): 374 (39, M+1); 300 (10, M-CHCOOH(NHBoc)+1); 91 (100, Bn)

HRMS (FAB): for C₂₁H₂₀N₅O₂ calculated 374.1617; found 374.1660.

¹H NMR (200 MHz, DMSO-d₆): 3.21 (m, 2H, CH₂); 4.28 (t, 1H, J=(CHCO, CH₂)=6.9, CHCO); 5.55 (s, 2H, CH₂Ph); 7.29-7.41 (m, 5H, arom.); 7.50 (d, 2H, J=8.3, arom.); 8.41 (v br, NH₃⁺); 8.79 (d, 2H, J=8.3, arom.); 8.85 (s, 1H, H-8); 9.01 (s, 1H, H-2).

¹³C NMR (125.8 MHz, DMSO-d₆): 36.06 (CH₂CH); 46.76 (CH₂Ph); 53.29 (CHCH₂); 117.44 (q, J=298.8, CF₃COOH); 127.97, 128.20, 129.01, 129.79 and 130.10 (CH-arom.); 130.47 (C-5); 134.59 (C-arom.); 136.72 (C-arom.); 138.45 (C-arom.); 146.78 (CH-8); 152.24 (CH-2); 152.54 and 152.68 (C-4 and C-6); 158.41 (q, J=31.5, CF₃COOH); 170.62 (CHCOOH).

IR (KBr): 3434, 3035, 2938, 1679, 1583, 1515, 1498, 1454, 1401, 1326, 1204, 1138, 836, 800, 722, 699.

Anal. calculated for C₂₃H₂₀F₃N₅O₄ (487.4): C, 56.67%; H, 4.14%; N, 14.37%; found: C, 56.42%; H, 4.14%; N, 14.14%.

### Example 30

### Production of (R,S)-2-amino-3-[4-(9-benzylpurin-6-yl)phenyl]propionic acid hydrochloride

(R,S)-3-[4-(9-Benzylpurin-6-yl)phenyl]-2-[(tert-butoxycarbonyl)amino]propanoic acid (75 mg, 0.16 mmol) obtained in Example 28 was dissolved in ethyl acetate saturated with hydrochloric acid (aprox. 1.7 M). The reaction mixture was stirred at room temperature for 3 hr, then the formed precipitate was filtered and recrystallized from methanol/ethyl acetate to give the title compound (65 mg, yield 100%) as white crystals.

¹H NMR (200 MHz, DMSO-d₆): 3.26 (d, 2H, J=6.1, CH₂CH); 4.25 (m, 1H, CHCH₂); 5.57° (s, 2H, CH₂Ph); 7.30-7.39 (m, 5H, arom.); 7.53 (d, 2H, J=8.3, arom.); 8.59 (br, NH₃⁺); 8.77 (d, 2H, J=8.3, arom.); 8.90 (s, 1H, H-8); 9.02 (s, 1H, H-2).

¹³C NMR (50.3 MHz, DMSO-d₆): 35.86 (CH₂CH); 46.83 (CH₂Ph); 53.20 (CHCH₂); 128.00, 128.24, 129.04, 129.85 and 130.20 (5×CH-arom.); 130.42 (C-5); 134.07 (C-arom.); 136.67 (C-arom.); 138.75 (C-arom.); 147.11 (CH-8); 151.97 (CH-2); 152.42 and 152.66 (C-4 and C-6); 170.51 (COO).

### Example 31

### Production of (S)-2-amino-3-[4-(9-benzylpurin-6-yl)phenyl]propionic acid hydrochloride

Dioxane/water (2:1, 5 ml) was added through septum to an argon purged flask containing 9-benzyl-6-chloropurine (59 mg, 0.24 mmol), (S)-4-boronophenylalanine (63 mg, 0.3 mmol), K₂CO₃ (88 mg, 0.64 mmol) and Pd(PPh₃)₄ (23 mg, 0.02 mmol). The mixture was stirred at 90° C. for 4 hr, diluted with water (40 ml) and pH of the solution was adjusted with aqueous HCl (2%) to 4. The mixture was washed with ethyl acetate and the solvent was evaporated in vacuo from the water part. The solid 15, residue was dissolved in water (1.5 ml) and crystallized from the solution at room temperature during 48 hr to give the title compound (50 mg, yield 61%) as white crystals.

¹H NMR (400 MHz, DMSO-d₆): 3.15 (dd, 1H, J=7.0 and 14.1, CH_{A}H_{B}CH); 3.26 (dd, 1H, J=5.4 and 14.1, CHAH_{B}CH); 3.95 (t, 1H, J=6.1; CHCH₂); 5.54 (s, 2H, CH₂Ph); 7.30-7.40 (m, 5H, arom.); 7.50 (d, 2H, J=8.2, arom.); 8.75 (d, 2H, J=8.2, arom.); 8.82 (s, 1H, H-8); 8.96 (s, 1H, H-2).

¹³C NMR (100.6 MHz, DMSO-d₆): 35.75 (CH₂CH); 46.48 (CH₂Ph); 53.18 (CHCH₂); 127.67, 127.90, 128.71, 129.46 and 139.80 (5×CH-arom.); 130.19 (C-5); 134.23 (C-arom.); 136.42 (C-arom.); 138.45 (C-arom.); 146.44 (CH-8); 151.94 (CH-2); 152.26 and 152.46 (C-4 and C-6); 170.16 (CO).

### INDUSTRIAL APPLICABILITY

The (purin-6-yl)amino acid of the present invention per se is useful as a pharmaceutical product such as an anti-cancer agent, antiviral agent and the like, or a production intermediate therefor, and the (purin-6-yl)amino acid can be produced easily according to the method of the present invention.

This application is based on patent application No. 2003-115403 filed in Japan, the contents of which are hereby incorporated by reference.

1. A (purin-6-yl)amino acid represented by formula (1) 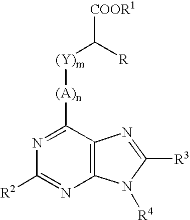
wherein R¹ is hydrogen, alkyl, optionally substituted aryl, optionally substituted heteroaryl or aralkyl; R² and R³ are hydrogen, halogen, optionally substituted alkyl, optionally substituted aryl, optionally substituted heteroaryl, optionally substituted amino or optionally substituted hydroxy; and R is —NH₂, —NHR′ or —NR′R″, said R′ and R″ are protecting group for amino group. Y is alkylene, alkenylene or alkynylene; A is optionally substituted phenylene; m and n are 0 or 1; and R⁴ is hydrogen or organic group,
or its salt.

2. The (purin-6-yl)amino acid according to claim 1, which is represented by formula (2): 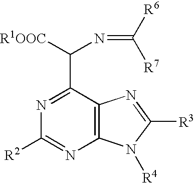
wherein R¹, R², R³ and R⁴ are as defined above; and R⁶ and R⁷ are optionally substituted aryl,
or its salt.

3. The (purin-6-yl)amino acid according to claim 1, which is represented by formula (3): 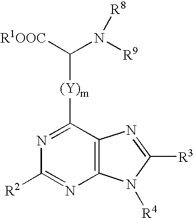
wherein R¹, R², R³, R⁴, Y and m are as defined above; and R⁸ and R⁹ are hydrogen or protecting group for amino group,
or its salt.

4. The (purin-6-yl)amino acid according to claim 3, wherein m is 1 and Y is methylene,
or its salt.

5. The (purin-6-yl)amino acid according to claim 3, wherein m is 1 and Y is trimethylene,
or its salt.

6. The (purin-6-yl)amino acid according to claim 3, wherein m is 1 and Y is propynylene, which is represented by formula (4): 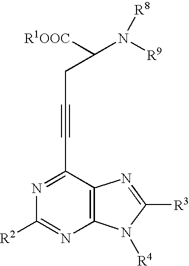
wherein R¹, R², R³, R⁴, R⁸ and R⁹ are as defined above,
or its salt.

7. The (purin-6-yl)amino acid according to claim 1, which is represented by formula (5): 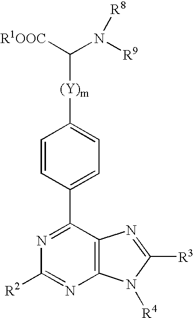
wherein R¹, R², R³, R⁴, R⁸, R⁹, Y and m are as defined above,
or its salt.

8. The (purin-6-yl)amino acid according to claim 7, wherein m is 1 and Y is methylene,
or its salt.

9. A synthetic method of the (purin-6-yl)amino acid described in claim 2, which is made a halogenated purine compound represented by formula (6): 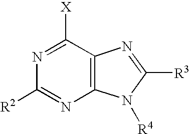
wherein X is halogen atom; and R², R³ and R⁴ are as defined above;
to react with an amino acid derivative represented by formula (7): 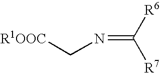
wherein R¹, R⁶ and R⁷ are as defined above.

10. A synthetic method of the (purin-6-yl)amino acid described in claim 3, which is made the halogenated purine compound represented by formula (6) to react with a halogenated amino acid derivative represented by formula (8): 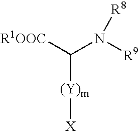
wherein R¹, R⁸, R⁹, X, Y and m are as defined above.

11. A synthetic method of the (purin-6-yl)amino acid described in claim 5, which is made the halogenated purine compound represented by formula (6) to react with an amino acid represented by formula (9): 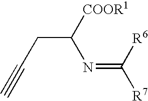
wherein R¹, R⁶ and R⁷ are as defined above.

12. A synthetic method of the (purin-6-yl)amino acid described in claim 7, which is made the halogenated purine-compound represented by formula (6) to react with an amino acid compound represented by formula (10): 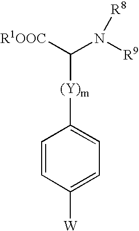
wherein R¹, R⁸, R⁹, Y and m are as defined above; W is —Sn(R⁵)₃, —B(OH)₂, —B(OR⁵)₂ or —MgX; R⁵ is lower alkyl; and X is as defined above.
